(19) United States
(12) Patent Application Publication
(10) Pub. No.: US 2009/0016995 A1
(43) Pub. Date: Jan. 15, 2009

(54) Method of Treating Cognitive Decline and Synaptic Loss Related to Alzheimer's Disease

(75) Inventors: Douglas W. Ethell, Riverside, CA (US); Gary W. Arendash, Lutz, FL (US)

Correspondence Address: SMITH HOPEN, PA, 180 PINE AVENUE NORTH, OLDSMAR, FL, 34677, US

(73) Assignees: UNIVERSITY OF SOUTH FLORIDA, Tampa, FL (US); REGENTS OF THE UNIVERSITY OF CALIFORNIA, Oakland, CA (US)

(21) Appl. No.: 11/934,940

(22) Filed: Nov. 5, 2007

Related U.S. Application Data
(63) Continuation of application No. PCT/US2006/016964, filed on May 3, 2006.
(60) Provisional application No. 60/677,180, filed on May 3, 2005.

(51) Int. Cl. A61K 35/26; A61P 25/28

(52) U.S. Cl. 424/93.7

(57) ABSTRACT

A method treating a subject with Alzheimer's disease wherein Aβ-reactive immune cells, splenocytes and lymphocytes, are generated in vitro or in vivo, re-stimulated in vitro, and then adoptively transferred into the cognitively-impaired subject. The population of immune cells can be derived from a donor with a natural or adaptive immune response to Aβ, stimulated by exposure to Aβ in vivo. The donor cells are then collected and re-stimulated by subsequent exposure to Aβ in vitro prior to administration to the subject. Alternatively, the population of immune cells can be derived from the subject and then stimulated by exposure to Aβ in vitro.

### CROSS REFERENCE TO RELATED APPLICATIONS

This application is a continuation of prior filed International Application, Serial Number PCT/US06/16964, filed May 3, 2006 which claims a priority date of May 3, 2005 based on prior filed U.S. Provisional Patent Application 60/677,180.

### FIELD OF INVENTION

This invention relates to a method of treating Alzheimer's disease.

### BACKGROUND OF THE INVENTION

The production of amyloid-β (Aβ) and it's deposition into neuritic plaques are central to the pathogenesis of Alzheimer's disease (AD) (Tanzi and Bertram, 2005). Active and passive immunization against Aβ have both been reported to reduce Aβ levels and deposition in the brains of AD transgenic mice, as well as provide cognitive benefits (Schenk et al., 1999; Bard et al., 2000; Morgan et al., 2000; Dodart et al., 2002). Soon after the first reports showing that Aβ vaccinations could improve cognitive deficits in mouse models, clinical trials were started with AD patients receiving Aβ vaccinations with adjuvant. Those trials, unfortunately, were halted due to the development of meningoencephalitis in some patients, apparently from immune over-activation, and the related death of at least one recipient (Orgogozo et al., 2003; Ferrer et al., 2004).

The ability of Aβ to cross the blood-brain barrier further complicates the active immune approach as it may seed Aβ fibril formation, and thus increase inflammation or neurotoxicity (Sigurdsson et al., 2002). Alternatively, several groups have administered Aβ-specific antibodies to confer passive immunity to AD transgenic mice (Bard et al., 2000). Passive immunity does improve cognitive performance and decrease brain Aβ levels (Dodart et al., 2002), but it has been reported to cause meningoencephalitis and increase cerebral amyloid angiopathy (CAA), along with associated microhemorrhages (WIlcock et al., 2004a,b; Lee et al., 2005).

Moreover, chronically high Aβ levels have been shown to result in immune hyporesponsiveness to Aβ in mice (Monsonego et al., 2001), which has led to suggestions that immune tolerance or anergy to Aβ may actively inhibit mechanisms that would normally lower Aβ levels in the brain (Ethell et al., 2002, 2003; Monsonego et al., 2003).

Therefore, what is needed is a method of immunotherapeutic intervention in AD to provide a limited and Aβ-specific activation or re-activation of the immune system, without inducing wider inflammatory responses from adjuvant.

Moreover, both active and passive immune approaches require regular boosters—monthly for active immunization and weekly for passive immunization. Thus, the needed method of activation of immune responses to Aβ must overcome theses serious complications that limit the translation of the prior art into clinical settings.

### SUMMARY OF INVENTION

Here, the inventors show that enhancing a T-cell immune response to Aβ overrides tolerance and provides beneficial effects. The inventive method transfers T-cell enriched populations of Aβ-specific immune cells, splenocytes and lymphocytes, into a cognitively-impaired subject suffering from AD-like cognitive impairment and pathology.

These significant cognitive and pathological benefits occur without triggering meningoencephalitis or enhancing CAA. Therefore, the inventive method of adoptive transfer of Aβ-responsive infusates provides a non-invasive and safe treatment for AD that can be administered at relatively infrequent intervals.

In one embodiment, the present invention includes a method for the treatment of a subject with Alzheimer's disease, comprising the step of administering a therapeutically effective amount of amyloid-beta specific immune cells to the subject. This embodiment is multi-facetted; variations on this embodiment are as follows.

In one variation of this embodiment the immune cells are T-cells; such as Th2-cells. The immune cells can be selected from the group consisting of lymphocytes and splenocytes.

In an alternate embodiment the amyloid-beta specific immune cells are exposed to a peptide constituent of amyloid plaque prior to administration to the subject. In varying embodiments the peptide constituent of amyloid plaque to which the immune cells are exposed is selected from the group consisting of amyloid-beta 1-42 and amyloid beta 1-40; or a combination thereof. Exposure can occur in vitro or in vivo. Variations also include embodiments wherein the immune cells are derived from a donor having an immune response to amyloid-beta or from the subject.

### BRIEF DESCRIPTION OF THE DRAWINGS

For a fuller understanding of the invention, reference should be made to the following detailed description, taken in connection with the accompanying drawings, in which:

FIG. 1A is a graph showing impaired RAWM working memory of 8-month old Tg mice prior to injection (*p<0.02 vs. non-Tg group). T1, semi-random first trial; T4+T5, combined working memory trials.

FIG. 1B is a graph showing pre- vs. post-injection RAWM working memory performance for each group across 3 blocks of testing (*p<0.05 or higher level of significance vs. pre-infusion errors). Tg/T cell mice, but not Tg/PBS controls, showed improved post-injection performance. For this, and all other behavioral graphs of this figure, behavioral testing occurred at 1-1½ months following a single injection.

FIG. 1C is a graph showing pre-infusion (final block) vs. post-infusion (first block) working memory. Tg/PBS control mice were impaired post-infusion (p<0.05, paired t-test) compared to pre-infusion, but not Tg/T-cell mice.

FIG. 1D is a graph showing superior working memory of Tg/T-cell mice vs. Tg/PBS mice on working memory trials T4+T5 during the first post-infusion block [*Tg/PBS group significantly impaired vs. both Tg/T-cell (p<0.05) and non-Tg (p<0.02) groups].

FIG. 1E is a graph showing the strong correlation between total hippocampal Aβ burden and working memory impairment in Tg improvement was seen in Tg/Oval or Tg/CD4− mice in the RAWM, when compared with Tg/PBS controls.

FIG. 1F is a graph showing the inability of ovalbumin-sensitized T-cell infusate (Tg/Oval) or T-cell depleted infusate (Tg/CD4−) to improve overall working memory (T4+T5) of impaired Tg mice (*p<0.02 for Tg/Oval and Tg/CD4− vs. non-Tg). Thus, neither non-specific protein stimulation or the fluid portion of the infusate were able to improve working memory of impaired Tg mice.

FIG. 1G is a graph showing significantly better spontaneous alternation performance of Tg/T-cell mice compared to Tg/PBS mice (*p<0.01 or higher significance vs. both other groups). Thus, Tg/T-cell mice had superior basic mneumonic processing vs. Tg/PBS controls.

FIG. 1H is a graph showing blood plasma levels of IL-1α, IL-1β, TNF-α and IFN-γ were similar in T-cell mice compared with Tg/PBS controls. All error bars represent SEM. Thus, the behavioral benefits of T-cell infusion occurred without inducing a global immune response at 1½ months post-injection.

FIG. 2A is a graph showing pre-infusion impaired working memory of Tg mice (a group different from those depicted in FIG. 1A) as indicated by increased errors during T4+T5 over 12 days of RAWM testing (*p<0.01 vs. non-Tg).

FIG. 2B is a graph showing a comparison of their RAWM working memory on Block 3 (last block) of post-infusion testing vs. pre-infusion testing (*p<0.05 or higher level of significance vs. pre-infusion errors). Only Tg mice given T-cells were able to reduce their number of working memory errors. (C & D) RAWM working memory for the last post-infusion block. For this graph and all other behavioral graphs of FIG. 2, behavioral testing occurred at 2-2½ months following a single injection.

FIG. 2C is a graph showing a comparison of RAWM working memory on the last post-infusion day of testing. Performance of Tg/T cell mice was not different from non-Tg controls, while the remaining Tg groups were impaired vs. non-Tg controls (*p<0.05 or higher level of significance vs. non-Tg group).

FIG. 2D is a graph showing RAWM working memory testing for the last post-infusion block (*p<0.05 or higher level of significance vs. non-Tg group for T4+T5; ‡p<0.02 vs. Tg/PBS group and no different from non-Tg group). Working memory performance of Tg/T-cell mice was equivalent to that of non-Tg controls. Since there were no group differences in T1 errors for C and D, the mean±SEM of all 4 groups is indicated for T1.

FIG. 2E is a graph showing overall performance in platform recognition. The superior search/identification ability of Tg mice given a T-cell injection 2½ months earlier is clearly evident (*p<0.002 vs. non-Tg mice; ‡p<0.05 vs. Tg/PBS and no different from non-Tg group). All error bars represent SEM.

FIG. 3A is a graph showing ELISA analysis of soluble Aβ1-42 in hippocampal extracts 2½ months after T-cell injection. Samples from Tg/T-cell mice showed a significant (*p<0.05) reduction in soluble Aβ1-42 when compared with Tg/PBS.

FIG. 3B is a graph showing soluble Aβ1-40 levels from hippocampal extracts 2½ months after T-cell injection. A non-significant decrease was seen in the Tg/T-cell group due to variability within extract readings.

FIG. 3C is a graph showing percentages of areas covered by lectin-stained microglia that surrounded plaques within the hippocampus (and dentate gyrus) 2½ months after injection (25 μm sections). Thus, Tg/T-cell mice exhibited a decrease in activated microglia.

FIG. 3D is a graph showing percentage of hippocampal area covered by lectin-stained microglia surrounding plaques 1½ months after adoptive transfer (12 μm sections). Error bars represent SEM. For a-d, asterisks indicate significant difference vs. Tg/PBS group at p<0.05, by Student's t-test. As at the 2½ time point above, Tg/T-cell mice also showed a decrease in activated microglia at 1½ months post-injection.

FIG. 4A is a graph showing IL-1α, IL-1β, TNF-α and IFN-γ levels in blood plasma showed no elevation in T-cell recipients 2½ months after transfer, compared with Tg/PBS controls (* significant difference from non-Tg mice, p<0.05). Thus, even through 2½ months post-injection, T-cells did not induce a global immune response.

FIG. 4B is a graph showing cytokine levels from hippocampal extracts showed elevations in TNF-α and IFN-γ in T-cell treated mice that were similar to non-Tg levels, but the differences between Tg/T-cell mice and Tg/PBS mice were not statistically significant.

FIG. 4C is a graph showing pro-inflammatory cytokines were not elevated in parietal cortex extracts of Tg/T-cell mice. All error bars represent SEM.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

### Donor Population

The donor population consisted of APPsw+PS1 transgenic (Tg) mice, derived from a cross between heterozygous male mice carrying the mutant APPK670N, M671L gene and heterozygous female mice bearing the mutant PS1 transgenic line 6.2. Offspring of this cross resulted in F2 generation Tg mice used in this study, which had the following mixed background: 56% C57, 12.5% B6, 19% SJL, and 12.5% Swiss Webster. Mice used for the ovalbumin, and T-cell depleted studies, discussed below, were derived from the F3 generation.

F3 generation APPsw+PS1 transgenic mice were derived from inbreeding the F2 generation Tg+ mice, preserving the same mixed background as the F2 mice. F3 APPsw+PS1 mice were generated from a cross between either heterozygous APPsw mice and heterozygous PS1 mice, or from a cross between APPsw+PS1 mice and non-transgenic mice.

Non-transgenic littermate mice, with the identical mixed background for all 3 studies, were used as controls. All mice were genotyped at weaning and housed in individual cages thereafter. Mice were maintained on a 12-h light/dark cycle, with free access to rodent chow and water. Behavioral testing was performed during the light period and in the same room where animals were being housed.

### Donor Preparation

Specific immune responses were generated in donor mice (littermates), lacking the APPsw (TgN2576) transgene, by vaccinating them with Aβ1-42 in complete Freund's adjuvant (CFA). Ten days later primary splenocytes and lymphocytes were isolated and re-stimulated in vitro with Aβ1-42 for 4 days to produce a T-cell enriched infusate. Those Aβ-responsive T-cells were then transferred into 8½ month old recipient Tg mice that displayed impaired working memory during the prior two weeks of testing in the radial arm water maze (RAWM) task. Treated and control mice underwent a battery of behavioral tests in two separate experiments, beginning either 1 or 2 months after this single injection.

The battery of tests consisted of post-infusion RAWM testing, as well as platform recognition, Y-maze, and a neurologic assessment screen (given in that order). Following completion of behavioral testing at 1½ or 2½ months post-treatment, mice were sacrificed for determination of anti-Aβ antibody titers, cytokine profiles, and histopathological analysis.

### Adoptive Transfer

Several non-transgenic littermates to the APPsw+PS1 subjects were selected as donors and vaccinated to generate adaptive immune responses to Aβ. Briefly: the backs of mice were shaved 1 day prior to injection. The following day mice were anaesthetized with isofluorane and given endodermal injections at three sites on the shaved backs. Each animal received 250 μg of monomeric human Aβ1-42 peptide (Bachem) in 100 μl of complete Freund's adjuvant (CFA) evenly divided in 3 sites. Although Aβ is usually ‘matured’ into fibrillar form for in vitro neurotoxicity (Ethell et al., 2002) and other vaccination studies (Schenk et al., 1999; Morgan et al., 2000), antigen presenting cells must proteolytically cleave it for presentation on MHCII. Difficulties in processing fibrillar Aβ may reduce the spectrum of possible antigenic peptides presented. In one set of mice, ovalbumin was substituted for Aβ in the entire protocol. Mice received booster injections of 200 ng pertussis toxin the following day and 2 days hence (i.p.). Mice were monitored for signs of distress and paralysis for 10 days, when they were sacrificed to harvest the spleen and draining lymph nodes. Others have reported paralysis and signs of encephalomyelitis after vaccination with Aβ using a similar protocol (Furlan et al., 2003), but most mice were sacrificed 2-4 days before such symptoms might usually be expected to appear (12-14 days after injection), and no paralysis was seen in mice sacrificed up to 1 month after Aβ vaccination.

Tissues from 3 mice were pooled and homogenized with a loose fitting 15 mL dounce until translucent (5-8 strokes). The mixture was passed over a 70 μm sieve filter, pelleted and resuspended in red blood cell lysis buffer for 5 minutes Cells were then gently pelleted, resuspended in full media, and counted. Cells were cultured at 1-2×106/mL in full media for 4 days with 8-10 μg/mL Aβ1-42, with some cultures also receiving 5 ng/mL IL-12 to enrich for Th1 cells. After 4 days in culture, cells were pelleted, resuspended in PBS, and viable cells counted by trypan blue exclusion.

Cognitively impaired APPsw+PS1 recipient mice received 5-20×106 viable cells in 0.5 mL PBS by tail vein injection (Tg/T-cell). Control APPsw+PS1 mice and non-transgenic mice received a tail vein injection of 0.5 mL PBS (Tg+/PBS and Tg− respectively). As additional controls, some APPsw+PS1 mice received a tail vein infusate of ovalbumin-sensitized immune cells or a T-cell depleted infusate. The former infusate controlled for the possibility that a nonspecific immune response to any generalized protein could provide cognitive benefit, while the later infusate controlled for the possibility that non-T-cell components (e.g., dendritic cells, monocytes, etc.) present within the complete infusate could provide cognitive benefit.

Recipient animals were monitored daily for signs of discomfort or paralysis. During cognitive testing, there were no visible sensorimotor differences between T-cell (or Th1-enriched) versus PBS-injected transgenic mice. In an extensive neurologic screen performed following completion of cognitive testing in both studies, there were no differences between transgenic groups across 17 measures including tail elevation, pelvic elevation, and hypotonic gait assessment.

### Behavioral Testing

Detailed methods for all three cognitive-based tasks have been described previously (Jensen et al., 2005). A brief description of each task is provided below: For the radial arm water maze task of spatial working memory, an aluminium insert was placed into a 100 cm circular pool to create 6 radially-distributed swim arms emanating from a central circular swim area. An assortment of 2-D and 3-D visual cues surrounded the pool.

The number of errors prior to locating which one of the 6 swim arms contained a submerged escape platform (9 cm diameter) was determined for 5 trials/day over 9-12 days of testing; 3-day blocks were used to facilitate statistical analysis. There was a 30 minutes time delay between the 4th trial (T4; final acquisition trial) and 5th trial (T5; memory retention trial).

The platform location was changed daily to a different arm, with different start arms for each of the 5 trials semi-randomly selected from the remaining 5 swim arms. During each trial (60 seconds max.), the mouse was returned to that trial's start arm upon swimming into an incorrect arm and the number of seconds required to locate the sub-merged platform was recorded. If the mouse did not find the platform within a 60 seconds trial, it was guided to the platform for the 30 seconds stay. The number of errors during Trials 4 and 5 are both considered indices of working memory and were thus averaged in this study. Analysis of the number of seconds taken per arm choice (an index of swim speed) over all days of post-treatment testing revealed no differences in swim speed between Tg groups in both Studies 1 and 2.

The platform recognition task measures the ability to search for and identify/recognize a variably-placed elevated platform. Because it was run immediately following the RAWM task and in the same pool, the platform recognition task requires animals to ignore the spatial cues present around the pool and switch from a spatial to an identification/recognition strategy—it is not a task of visual acuity alone in our paradigm, with cognitive-based performance directly linked to levels of brain Aβ (Leighty et al., 2004). Mice were given four successive trials per day over a 3-4 day period. Latencies to find an elevated platform (9 cm diameter), bearing a prominent cone-shaped styrofoam ensign on a wire pole, were determined. For each trial (60 seconds max.), animals were placed into the pool at the same location and the platform was moved to a different one of four possible locations. For statistical analysis, escape latencies for all four daily trials were averaged.

The Y-maze task of spontaneous alternation measures basic mnemonic function. Mice were allowed to explore a black Y-maze with 3 arms for 5 minutes, with the number and sequence of arm choices being recorded. Percent spontaneous alternation (the ratio of arm choices differing from the previous two choices divided by the total number of entries) was determined for each animal.

For statistical analysis of RAWM data, both one-way ANOVAs and two-way repeated measure ANOVAs were employed. For platform recognition and Y-maze spontaneous alternation, one-way ANOVAs were used. Following ANOVA analysis, post hoc pair-by-pair differences between groups were resolved with the Fisher LSD test.

### Pathological Processing

After behavioral testing, animals were deeply anesthetized, their chest cavity opened, and an intra-cardial blood sample was taken. Following intracardial perfusion with PBS, brains were excised and divided in two (mid-sagital). In the first study (1½ months post-infusion) one half of each brain was immersed in Bouin's fixative for 4 days and the other half was fixed in 4% paraformaldehyde, followed by graded sucrose solutions.

Bouin's fixed samples were cryoprotected with increasing sucrose concentrations, then 15 μm sections were made on a cryostat, and floated into PBS. In the second study (2½ month post-infusion), following PBS perfusion, brains were not fixed immediately. Rather, half of each brain was micro-dissected to isolate parietal cortex and hippocampus, which were later utilized for cytokine and soluble/insoluble Aβ analyses. The other half of each brain from the 2½ month experiment was snap frozen, and a cryostat was used to make 25 μm coronal sections that were mounted onto charged slides and dried for 10-15 min at 55° C. Sections mounted on half of the slides were fixed by immersion in cold acetone (−20° C.) for 10 min, and the other half fixed by immersion in Bouin's at room temp for 10 minutes Both fixation procedures were followed by repeated rinsing in PBS, and then dried until use.

### Thioflavin S and 6E10 Staining

Paraformaldehyde-fixed samples from the 1½ month set were sectioned at 25 μm for quantification of diffuse Aβ (immunostaining with 6E10 antibody) and compact Aβ (thioflavin S staining) according to the established protocol of Costa et al. (2004). Bouin's fixed sections from the 2½ month set were stained by immersion in 0.025% Thioflavin S in 50% EtOH/50% PBS at room temperature for 2 minutes Sections were then rinsed repeatedly with 50% EtOH/50% PBS, and finally PBS. For Thioflavin S staining analysis fluorescent images were captured using a Nikon 2000U fluorescence microscope fitted with a Spot video capture system. Images of 3 coronal sections (through the thalamus) from each mouse were analyzed using NIH ImageJ. Strong fluorescence of Thioflavin S staining allowed for the use of automatic threshold adjustment. Mean of all animals in each experimental group were compared and not found to be statistically different. Images of 6E10 staining were taken using visible light. Aβ loads as a percentage of total area stained with 6E10 were determined from hippocampus and adjacent parietal cortex in 5 coronal sections that included the thalamus.

### Staining for Microglia

For the 1½ month post-infusion time point, brain halves were fixed in Bouin's and cryoprotected by serial overnight immersions in 10% sucrose-PBS, then 30% sucrose-PBS. 15 μm coronal sections were made from each brain and 10 comparable sections near the caudal diencephalon were selected from each animal, and stained for microglia with biotinconjugated tomato lectin (5 μg/ml) in PBS supplemented with 0.1% BSA, 1% NGS, and 0.2% triton X-100. Subsequent incubations with conjugated markers, avidin-HRP (Vectastain ABC) or streptavidin-PE (BD Biosciences), were used for visualization. This method stains microglia and blood vessels with the two being morphologically distinguishable (Schmid et al., 2002). For the 2½ month post-infusion time point, half of each brain was snap frozen without fixative, and stored at −85° C. Frozen sections were made at 25 μm, mounted directly onto charged glass slides, and heated to 60° C. for 10-20 minutes Sections were stored dry, rehydrated by immersion in PBS for 5-10 minutes, and stained for lectin as above.

Images of HRP stained sections were captured by video microscopy. For both series, 3 sections from the majority of mice in each experimental group were analyzed for microglial staining around plaques, referred to here as, “microglia plaque shells”. Every plaque shell in each image was marked and the boundaries determined by edge contrast calculations using Adobe Photoshop's, “magic wand”. Composite images containing all discernable plaque shells in the hippocampus and dorso-lateral parietal cortex of these sections were saved as “jpeg and tiff” files. Boundaries of the hippocampus and adjacent cortex were set to determine the percent area covered by the scored microglia plaque shells using the analyze particle feature of NIH ImageJ.

### Soluble and Insoluble Aβ Analysis

For the 2½ month study, hippocampal levels of both soluble/insoluble Aβ1-40 and Aβ1-42 were measured by ELISA. Briefly, mouse hippocampal tissues were homogenized according to the protocol described by Schmidt et al. (2005a). Diethylamine (DEA) extractions were then performed on homogenates using the protocol described by Schmidt et al (2005b). The supernatants obtained from this protocol were then stored at −80° C. for later ELISA analysis of soluble Aβ levels. The pellets were re-suspended in sample diluent to original volume.

Formic acid extraction was then conducted on these suspensions and supernatants obtained were stored at −80° C., to be used for insoluble Aβ ELISA determinations. All extracts were thawed, appropriately diluted, then analyzed for both Aβ1-40 and Aβ1-42 levels using ELISA kits obtained from Signet laboratories.

### Cytokine Analysis

In Study 1 (1½ month), cytokine levels from plasma were determined using a custom RayBio Mouse Cytokine Antibody Array (RayBiotech, Inc.). In Study 2 (2½ month), cytokine levels from plasma, as well as from hippocampus and parietal cortex, were detected by using Bioplex (Bio-Rad, catalog 171F11181) kits according to the manufacturer's protocol. Because of the naturally-occurring large variability (100×-1000×) in optical density readings among the various cytokines, optical density readings for each cytokine were converted to standardized signal intensities. For this conversion, signal mean intensities minus background signal intensity were determined and standardized to a zero to one scale based on minimum and maximum individual intensity readings for each cytokine. These standardized values were then used for relative cytokine level comparisons among animal groups.

### Anti-Aβ Antibody ELISA

For determination of plasma anti-Aβ antibody levels from blood taken at euthanasia, 96 well plates were coated with 50 μl Aβ1-16 peptide in CBC buffer at 10 μg/ml. A CBC plate was set up for binding background, then both Aβ and CBC plates were incubated at 4° C. overnight. After 5 washes with wash buffer, plates were subjected to a blocking step with 180 μl blocking buffer (1×PBS containing 1.5% BSA), then washed an additional 5 times with wash buffer. Samples diluted with blocking buffer were then added into both Aβ plates and CBC-plates, with two-fold serial dilutions starting with 1:50, then incubated at 37° C. for 1 hour, followed by 12 washes with wash buffer. HRP-conjugated anti-mouse IgG was loaded into each well at 1:5000 dilution with dilution buffer, incubated for 1 hour at 37° C., then washed 12 times. TMB substrate was dissolved in PCB buffer and 100 μl was added into each well. Color reaction was stopped with 25 μl 2N H2SO4. Plates were read at 450 nm/620 nm, with those samples having readings three times higher than controls being considered as positive. For any given measure, initial one-way ANOVAs involving all groups were followed by post hoc pair-by-pair group differences using the Fisher LSD test.

### Synaptophysin Staining

Bouin's fixed, coronal sections from the 2½ month cadre were rehydrated in PBS for 30 minutes, then stained with Thioflavin S as above. Sections were re-equilibrated with PBS, then permeabilized with 0.1% triton x-100 in PBS for 10 minutes Sections were rinsed 3× with PBS and non-specific binding blocked with 1% BSA and 5% NGS in PBS for 90 minutes Sections were incubated with a 1:100 dilution of mouse anti-synaptophysin (SVP-38, Sigma) in PBS+0.05% tween-20 for 2 hours. Sections were then rinsed with PBS 3×10 min, then incubated with 1:500 Alexa680-conjugated goat anti-mouse secondary (Molecular Probes) for 30 minutes, then rinsed 3×5 minutes with PBS. Sections were mounted with DAPI-containing Vectashield (Vector Laboratories) and cytoseal. Confocal microscopy was done on a Zeiss LSM510 confocal microscope fitted with a 10× objective.

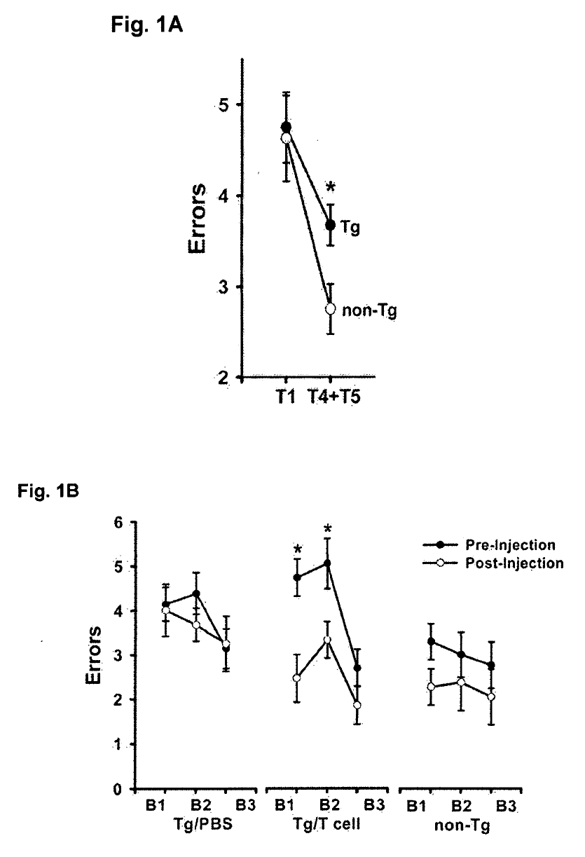

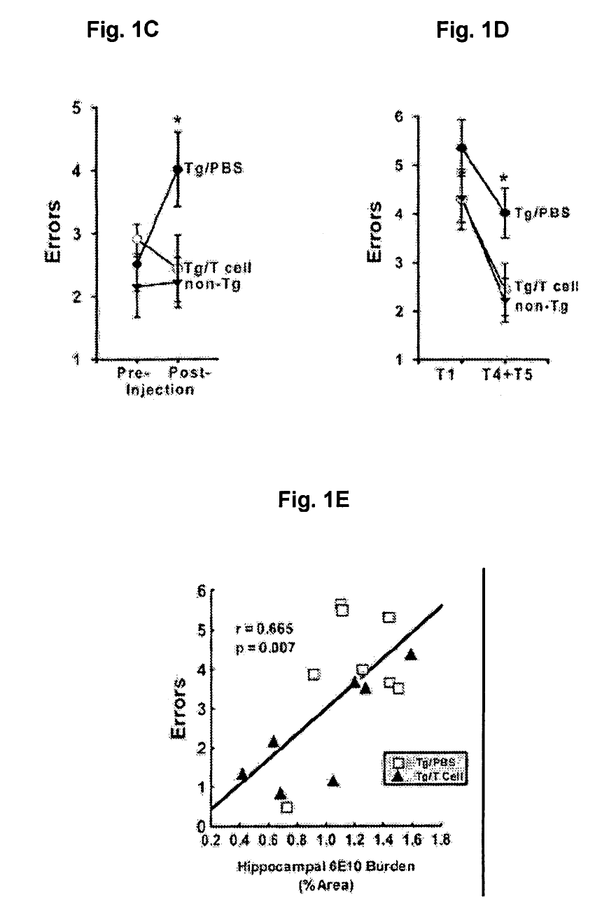

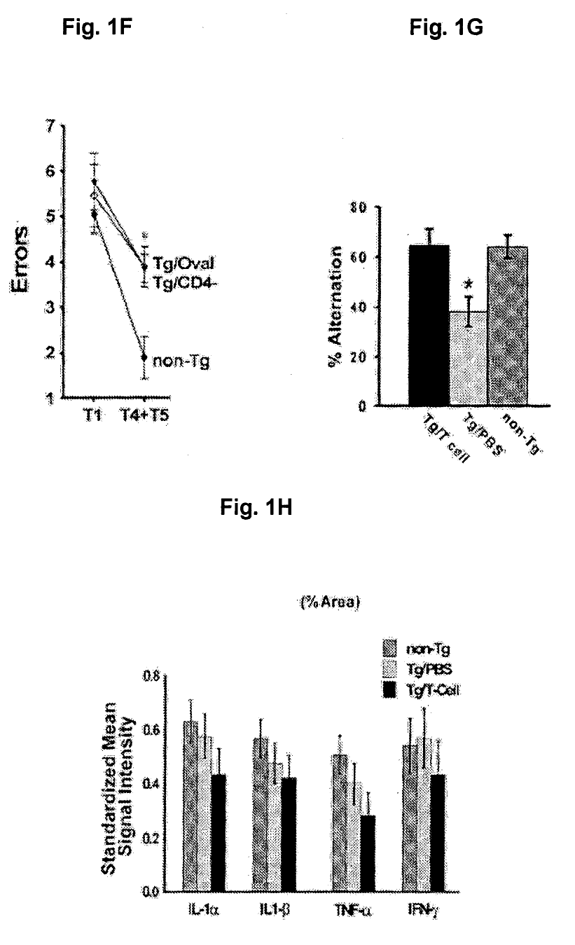

Fifteen transgenic (Tg) and 8 littermate non-Tg mice were selected for cognitive testing prior to adoptive transfer. At 8 months of age, the mice were subjected to working memory assessment in the radial arm water maze (RAWM) task (Leighty et al., 2004). Over 12 days of testing, Tg mice showed impaired working memory compared to non-Tg mice, as evidenced by a higher number of errors during Trials 4+5 overall (FIG. 1A). On the day following completion of pre-injection testing, Tg mice were divided into two groups equally balanced in working memory performance; animals in one Tg group received a single tail vein injection of T-cells (n=7), while animals in the other Tg group (n=8), and non-Tg mice, received a control PBS injection. Between 1-1½ months after adoptive transfer, animals were tested again in the RAWM task over three 3-day blocks, followed by platform recognition (4 days) and Y-maze (1 day) testing.

A comparison of pre- vs. post-injection working memory performance revealed no improvement for Tg/PBS mice overall or for individual blocks of testing (FIG. 1B). In sharp contrast, Tg/T-cell mice significantly improved their working memory performance (Trials 4+5) overall (p<0.01), particularly during the first two blocks of post-injection testing (FIG. 1B). Non-Tg mice were also able to improve their working memory performance, but not significantly because of relatively good pre-injection performance. Comparisons of the final block of pre-infusion testing to the first block of post-infusion testing showed that Tg/PBS mice became significantly worse (p<0.05) during initial post-infusion testing (FIG. 1C). By contrast, Tg mice that received T-cell infusions maintained their final pre-infusion performance level, as did non-Tg controls. Indeed, Tg/T-cell mice were no different in working memory performance compared to non-Tg controls. A significant block by treatment effect was present (p<0.02) due to the worsened performance of Tg/PBS mice during initial post-infusion testing compared to the other two groups. The immediate beneficial effects of T-cell infusion on working memory in Tg mice were further confirmed by evaluation of working memory (combined Trials 4+5) during the first block of post-infusion testing (FIG. 1D). As is typical, all three groups did not differ from one another during the semi-random Trial 1 (T1). However, performance during the working memory trials (T4+T5) indicated that Tg/T-cell mice performed identically to non-Tg controls and significantly better than Tg/PBS control mice (p<0.05).

With all Tg mice included, correlation analysis of RAWM working memory performance during the important first block of post-injection testing vs. hippocampal 6E10 staining of diffuse Aβ deposition revealed a strong positive correlation (FIG. 1E). Higher hippocampal Aβ burdens were associated with poorer working memory (p=0.007). Identifying each Tg mouse by whether or not it received T-cell infusion clearly showed that this strong correlation was driven by the Tg/T-cell group. Seven of 8 Tg/PBS mice were grouped together with higher Aβ burdens and poorer working memory performance (FIG. 1E). Although 3 Tg/T-cell mice were also in this cluster, the remaining 4 Tg/T-cell mice were clustered together with lower hippocampal Aβ burdens and superior working memory performance. Thus, there were two sub-groups of Tg/T-cell mice—one group that benefited greatly in working memory with substantially reduced hippocampal Aβ burdens, while the other group showed minimal behavioral benefit with no reduction in hippocampal Aβ burdens. Nonetheless, by the final post-infusion block of testing, even the latter group showed cognitive benefit compared to their performance during the initial block of post-infusion testing (2.5 vs. 3.8 errors; t=6.65; p=0.022). When these two subsets of Tg/T-cell mice were considered collectively, there were no significant reductions vs. Tg/PBS mice in either hippocampal or parietal cortex diffuse (6E10) Aβ deposition (0.97±0.13% vs. 1.18±0.12% for hippocampus and 1.52±0.24% vs. 1.55±0.22% for parietal cortex).

Similarly, no significant differences in compact (Thioflavin S-stained) Aβ burdens were evident between Tg/T-cell and Tg/PBS groups in either hippocampus or parietal cortex (1.53±0.11% vs. 1.50±0.10% for hippocampus and 1.69±0.14% vs. 1.65±0.13% for parietal cortex). Qualitative assessment of thioflavin S-stained brain sections revealed a limited amount of cerebral amyloid angiopathy in Tg mice, which was not noticeably increased in T-cell infused mice.

To determine if the responses observed were generalized immune responses we vaccinated a series of donors with chicken ovalbumin and prepared infusates with ovalbumin restimulation (Tg/Oval). Seven cognitively-impaired Tg mice received ovalbumin stimulated infusates, but none showed significant cognitive benefit 1-1½ months after transfer, as exemplified by the RAWM task (FIG. 1F). To determine if T-cells were necessary for the beneficial effects observed, we generated standard infusates with Aβ and depleted them of T-cells with CD4+ magnetic beads prior to infusion into cognitively impaired Tg mice (Tg/CD4−). CD4+ T-cell-depleted infusates provided no cognitive benefits to any of 7 cognitively-impaired Tg recipients 1-1½ months after transfer (FIG. 1F).

Although there were no group differences in platform recognition testing, Tg/T-cell mice performed identically to non-Tg mice and significantly better than Tg/PBS mice in the Y-maze task of spontaneous alternation (FIG. 1G), which evaluates basic mnemonic processes. Thus, a single T-cell infusion benefited cognitively-impaired Tg mice by improving both their working memory performance and basic mnemonic function to the levels of non-Tg controls. These benefits of adoptive T-cell transfer occurred without significant elevations in plasma levels of the pro-inflammatory cytokines IL-1α, IL-1β, TNF-α or IFN-γ (FIG. 1H); indeed, Tg/T-cell mice had generally reduced plasma cytokine levels.

Thus, adoptive T-cell transfer does not produce a global, sustained inflammatory response. Moreover, haematoxylin and eosin staining of brain sections revealed no evidence of meningoencephalitis (e.g., mononuclear inflammatory cells associated with the cerebrovasculature and leptomeninges overlying the cortex) in Tg/T-cell mice.

To determine if the aforementioned behavioral benefits were sustainable over an even longer period following T-cell treatment, we repeated the experiment on a separate cadre of 15 cognitively-impaired Tg mice, with behavioral evaluation between 2-2½ months post-infusion.

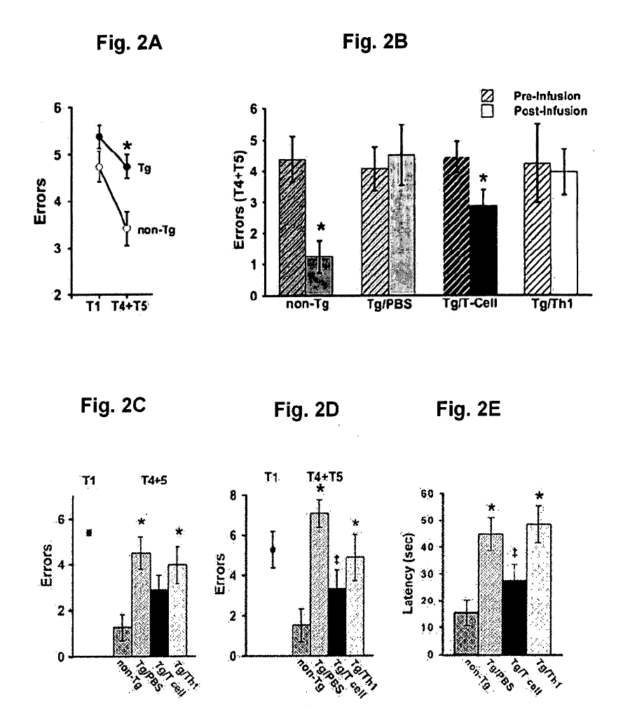

Further, to establish if the beneficial effects were impacted by Th1:Th2 ratios, cultures were re-stimulated with Aβ1-42 alone, or supplemented with IL-12, which causes a Th2 to Th1 shift. At 8½ months of age, Tg mice that were cognitively-impaired in RAWM working memory (FIG. 2A) received a single tail vein infusion of T-cells (n=6), Th1 enriched cells (n=4), or PBS (n=5). Age-matched non-Tg mice were given PBS (n=8) and run concurrently.

After 2-2½ months, Tg/T-cell mice again still had improved RAWM working memory, which occurred during the later phases of testing. This benefit is exemplified by comparing block 3 (last block) of post-infusion testing to block 3 of pre-infusion testing for each group (FIG. 2B). Of the three Tg groups, only Tg mice given a T-cell injection were able to reduce their number of working memory errors. Similarly, during both the final block (FIG. 2C) and the final day (FIG. 2D) of post-infusion RAWM testing, working memory of Tg/T-cell mice was not different from non-Tg controls and significantly better than Tg/PBS mice. Overall performance in the ensuing platform recognition task revealed a significant impairment in both Tg/PBS and Tg/Th1 mice at this later testing age. By contrast, Tg/T-cell mice were not impaired vs. non-transgenic controls, exhibiting superior strategy-shifting and search/identification skills compared to Tg/PBS (FIG. 2E). All groups performed similarly in the Y-maze spontaneous alternation task. Results from both the RAWM and platform recognition tasks indicate that the cognitive benefits of adoptive T-cell transfer transcend several cognitive domains, even after 2-2½ months.

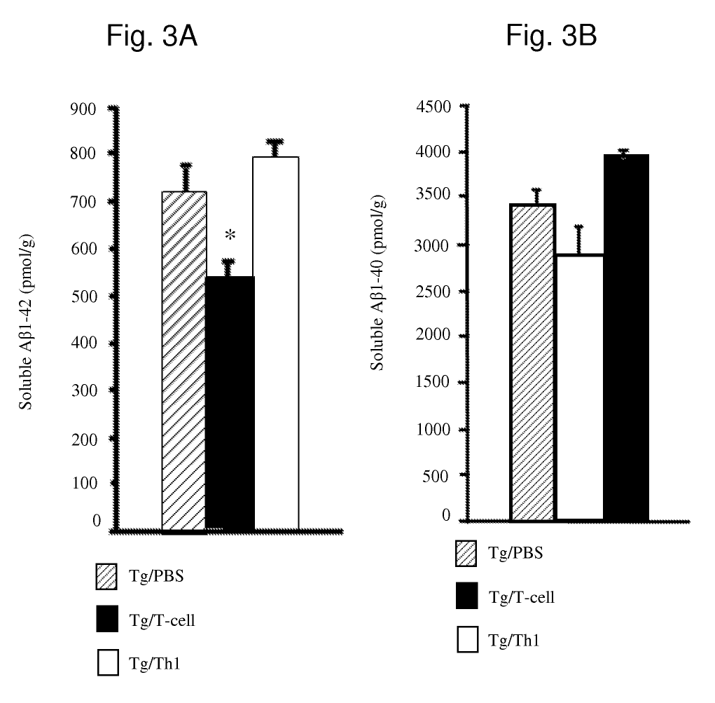

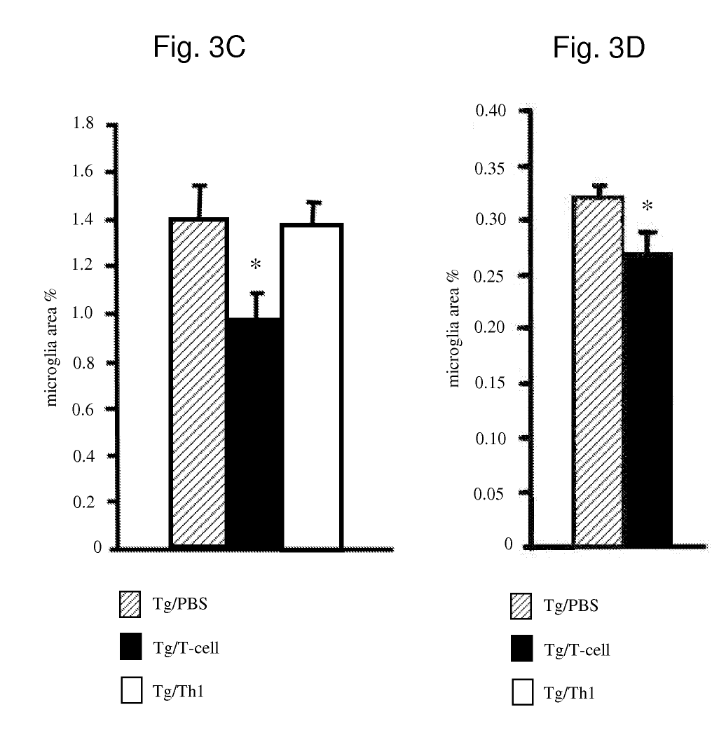

ELISA analysis of hippocampal extracts from Tg/T-cell mice after 2½ months showed a significant 25% reduction of soluble Aβ1-42 levels (FIG. 3A) and a nearly significant (p=0.09) reduction in Aβ1-40 levels compared to Tg/PBS mice (FIG. 3B). Although Tg/T-cell mice also showed downward trends for hippocampal levels of insoluble Aβ1-42 (↓29%) and insoluble Aβ1-40 (↓22%), the reductions were not statistically significant due to variability within groups. Thioflavin S staining showed similar Aβ burdens in the hippocampus of Tg/PBS (1.41+0.07%), Tg/T-cell (1.37+0.15%), and Tg/Th1 (1.28±0.12%) mice, that were not significantly different. At both the 1½ and 2½ month post-infusion time points, microglia surrounding dense deposits covered a significantly smaller area of the hippocampus in Tg/T-cell, compared with Tg/PBS or Tg/Th1 mice (FIG. 3C-D). Therefore, the hippocampus of Tg/T-cell mice had notably lower levels of soluble Aβ and plaque-associated microglia, but maintained high levels of insoluble and dense-core Aβ.

Measurement of anti-Aβ antibody titers in blood plasma 2½ months after adoptive transfer indicated that none of the Tg groups had significant anti-Aβ responses at multiple dilutions of plasma between 1:400 and 1:50. In a follow-up cohort of Tg mice wherein blood samples were taken at 2 weeks, 4 weeks, and 6 weeks following T-cell infusion, plasma anti-AP antibody titers were similarly very low at multiple plasma dilutions. Thus, any Aβ antibody response generated by T-cell adoptive transfer had subsided by two weeks post-infusion, though cognitive benefits were evident well beyond this point.

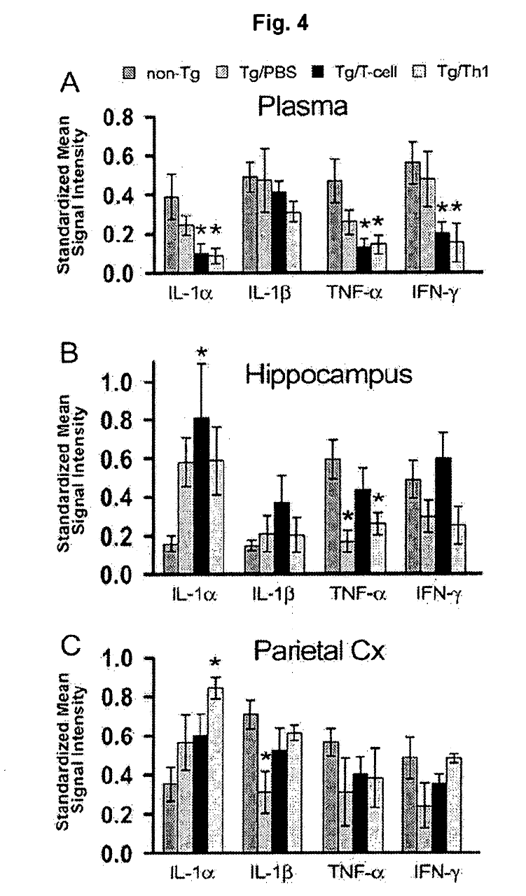

Consistent with results from the earlier 1½ month study, plasma levels of IL-1α, IL-1β, TNF-α and IFN-γ were similar or reduced in T-cell and Th1 infused Tg mice compared with Tg/PBS mice at the more extended 2½ months time point (FIG. 4A). Levels of these same cytokines in hippocampus and parietal cortex of Tg/T-cell and Tg/Th1 mice were also reduced or similar to non-Tg and/or Tg/PBS mice at 2½ months (FIGS. 4B and C). Thus, behavioral benefit to Tg/T-cell mice occurred independently of high anti-Aβ antibody titers or a sustained global inflammatory response.

The synaptotoxic effects of oligomeric Aβ have been shown to deleteriously affect both synaptic density and LTP in the hippocampus (Cullen et al., 1997; Mungarro-Menchaca et al., 2002; Costello et al., 2004). The present results of synaptophysin staining at 2½ months post-infusion revealed higher synaptic densities in the hippocampus of Tg/T-cell mice, compared with Tg/PBS or Tg/Th1 mice.

Synaptophysin staining in Tg/T-cell mice was similar to non-Tg mice. Interestingly, the proximity of dense-core deposits (Thioflavin S) did not appear to impact synaptic density, which is consistent with evidence that soluble forms of Aβ are more synaptotoxic than dense-core deposits (Walsh and Selkoe, 2003). Tg/Th1 also showed more hippocampal synaptophysin staining than Tg/PBS mice, although still much less than Tg/T-cell cohorts. The contrast between synaptophysin staining in Th1 and T-cell treated mice indicates that biasing immunogenic responses toward Th1 is less effective, and may represent only a slowing of the pathophysiology.

The present invention provides a method wherein a single transfer of Aβ-stimulated T-cells can reverse cognitive deficits and synaptic losses for at least 2½ months; as shown in the APPsw+PS1 mouse model for AD. Treated mice had lower levels of soluble Aβ in the hippocampus, yet retained high levels of insoluble Aβ and dense-core deposits. These findings support the consensus that early cognitive decline in AD results from the synaptotoxic activity of soluble Aβ intermediates (Kayed et al., 2003; Cleary et al., 2005; Klyubin et al., 2005).

Plasma and brain extracts both showed lower or normal (non-Tg) levels of pro-inflammatory cytokines in T-cell recipients 1½ and 2½ months after transfer, confirming that a sustained, widespread inflammatory response was not present at those time-points and was not necessary for continued beneficial effects. The cognitive benefits of T-cell transfer were associated with decreased numbers of plaque-associated microglial cells. Prior work in APPsw+PS1 mice has linked a decrease in microglial activity (through blockage of their CD40 receptors) to improved cognitive performance (Tan et al., 2002).

No evidence of meningoencephalitis was found; which can occur following both active and passive Aβ immunotherapy (Orgogozo et al., 2003; Lee et al., 2005). In view of the substantial increase in CAA reported in some studies following passive Aβ immunotherapy (Wilcock et al., 2004b), it is important to note that the limited CAA present in Tg mice was not exacerbated by T-cell infusion. Thus, adverse effects associated with both active and passive Aβ immunotherapies are not evident with the adoptive transfer of Aβ-specific T-cells.

In contrast to mice receiving T-cell (both Th1 and Th2 cell) transfers, those given Th1-enriched infusates showed minimal or no improvement in cognitive performance, microglial staining, or synaptic density. These results indicate that pro-inflammatory T-cell (Th1) responses may be expendable and that Th2 cells play a key role in this therapy, although a combination of Th1, Th2, and other cells may be required. As Th2 cells facilitate antibody responses, these findings are consistent with reports showing that Aβ-specific antibodies can improve cognitive performance and assist in the clearance of Aβ from the brains of AD transgenic mice (Dodart et al., 2002; Wilcock et al., 2004a,b). However, at several weeks through 2½ months following a single adoptive transfer of T-cells, Aβ-specific antibody titers in plasma were very low, even though the cognitive and pathophysiologic benefits continued. Additional mechanisms, other than anti-Aβ antibody formation (see below), could therefore be important for the long-term benefits observed with T-cell adoptive transfer in AD transgenic mice. The lack of cognitive benefit provided by ovalbumin-sensitized T-cells or T-cell-depleted infusates underscores that neither a generalized immune response or non-T-cell components of the infusate are capable of providing the benefits observed with Aβ-sensitized T-cells (both Th1 and Th2).

The cognitive recovery of mice through at least 2½ months shows the lasting impact of a single adoptive transfer treatment. This extended period of recovery involved an enhancement of multiple cognitive domains (working memory, recognition/identification, basic mnemonic processing). These two findings suggest that continuous treatments in AD patients may not be necessary to achieve a spectrum of cognitive-enhancing effects. Aβ-reactive T-cells can be isolated from an AD patient's blood, or even spleen, expanded in vitro and returned to the patient on a periodic basis.

These findings show that a restricted immune response to Aβ is sufficient to reverse cognitive impairment and lessen some brain pathology in AD transgenic mice over a surprisingly long period of time. Endogenous mechanisms repress immune responses to Aβ, while boosting Aβ-specific Th2 responses overrides that repression, which aids in lowering soluble Aβ within the brain without activating a global inflammatory response or inducing meningoencephalitis. Therefore, the adoptive transfer of Aβ-specific T-cells represents an efficacious approach to the treatment of Alzheimer's disease.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method for the treatment of a subject with Alzheimer's disease, comprising the step of administering a therapeutically effective amount of amyloid-beta specific immune cells to the subject.

2. The method of claim 1 wherein the immune cells are T-cells.

3. The method of claim 2 wherein the T-cells are amyloid-beta specific Th2 cells.

4. The method of claim 1 wherein the immune cells are selected from the group consisting of lymphocytes and splenocytes.

5. The method of claim 1 wherein the amyloid-beta specific immune cells are exposed to a peptide constituent of amyloid plaque prior to administration to the subject.

6. The method of claim 5 wherein the peptide constituent of amyloid plaque is selected from the group consisting of amyloid-beta 1-42 and amyloid beta 1-40.

7. The method of claim 5 wherein the amyloid-beta specific immune cells are exposed to the peptide constituent of amyloid plaque in vitro.

8. The method of claim 5 wherein the amyloid-beta specific immune cells are exposed to the peptide constituent of amyloid plaque in vivo.

9. The method of claim 1 wherein the amyloid-beta specific immune cells are derived from a donor having an immune response to amyloid-beta.

10. The method of claim 1 wherein the amyloid-beta specific immune cells are derived from the subject.

11. A method for the treatment of a subject with Alzheimer's disease, comprising the steps of:
obtaining a population of amyloid-beta specific T-cells;
exposing the amyloid-beta specific immune cells with a peptide constituent of amyloid plaque; and
administering a therapeutically effective amount of the amyloid-beta specific immune cells to the subject.

12. The method of claim 11 wherein the T-cells are amyloid-beta specific Th2 cells.

13. The method of claim 11 wherein the peptide constituent of amyloid plaque is selected from the group consisting of amyloid-beta 1-42 and amyloid beta 1-40.

14. The method of claim 11 wherein the amyloid-beta specific immune cells are exposed to the peptide constituent of amyloid plaque in vitro.

15. The method of claim 11 wherein the amyloid-beta specific immune cells are exposed to the peptide constituent of amyloid plaque in vivo.

16. The method of claim 11 wherein the amyloid-beta specific immune cells are derived from a donor having an immune response to amyloid-beta.

17. The method of claim 11 wherein the amyloid-beta specific immune cells are derived from the subject.

18. The method of claim 11 wherein the immune cells are selected from the group consisting of lymphocytes and splenocytes.
